(19) United States
(12) Patent Application Publication
(10) Pub. No.: US 2024/0046337 A1
(43) Pub. Date: Feb. 8, 2024

(54) HEALTHCARE SERVICE ORDER FULFILLMENT SYSTEM AND METHOD

(71) Applicant: Jeffrey KNIGHT, Buffalo, NY (US)

(72) Inventor: Jeffrey KNIGHT, Buffalo, NY (US)

(21) Appl. No.: 17/817,842

(22) Filed: Aug. 5, 2022

(51) Int. Cl. G06Q 30/06; G16H 20/10

(52) U.S. Cl. CPC G06Q 30/0637; G16H 20/10

(57) ABSTRACT

The inventive concept is a computerized healthcare product and service ordering system and method. At least one processor is designed to assess the completeness of service orders and contains information about ordered products, services, and associated administrative communications. The processor is programmed to distinguish between the product and service ordered and associated administrative communications, record the information, send confirmation, request fulfillment, and establish a repeat order queue, wherein the software program assesses the priority of the order and bypass trigger points. At least one processor is designed to receive order completeness information for fulfillment and to take corrective action if needed. The processor is designed to assess whether the order is fulfilled on time. The processor, if fulfillment is or will be late, at least one or more of quantitatively, qualitatively, and by delivery location incomplete, is designed to alert selected people. The processor is designed to verify order fulfillment.

### FIELD OF THE INVENTION

The inventive concept relates generally to a healthcare service order fulfillment system and method.

### BACKGROUND

Currently, handling healthcare orders includes a mix of manual and computer-supported systems where the possibility of making mistakes, creating inefficiencies, and missing deadlines is high. Further, such systems leave open the possibility of fraud and ease the opportunity to healthcare operators to favor profitability above the welfare of patients. Errors may go unnoticed that could otherwise have been detected. Documents may be altered without the opportunity to detect documents have changed. Further, even if a service provider has implemented efficiencies within their system, inefficiencies within those fulfilling orders may negate those created by the service provider. Coordinating fulfillment from service providers may be difficult to impossible. Therefore, there is a need in the market for an improved system for fulfilling healthcare orders for service providers.

### SUMMARY OF THE INVENTION

Disclosed is a healthcare product and service ordering system that has at least one computer processor adapted to receive an order via at least one user interface operable with at least one memory storage medium wherein the service order includes at least one or more of a product and a service. The at least one processor is designed to assess the completeness of the order. The order contains information about at least one or more of the product and service ordered and associated administrative communications. The processor is programmed to distinguish between the product and service ordered and associated administrative communications. The software program is designed to at least one or more of record the information, send confirmation, send the order for fulfillment, and establish a repeat order queue, wherein the software program may be disposed on at least one or more of a single processor and distributed system.

The at least one processor is designed to assess the priority of the at least one or more of a product and service ordered and, upon reaching a selectable priority bypass trigger point, is designed to select the order for fulfillment. The at least one processor is further designed to send incomplete information to the order source for completion. The at least one processor is designed to receive for fulfillment at least one or more of the completed order information from the software program and the order for the at least one or more product and service needed to fulfill the order. The processor is designed to monitor order fulfillment until fulfillment is completed.

The processor is designed to assess whether the order is fulfilled on time. The processor, if fulfillment is at least one or more of late, reaches a selected threshold prior to lateness, is determined will be late, and at least one or more of quantitatively, qualitatively, and by delivery location incomplete, is designed to alert at least one or more of a salesperson and a manager, adapted to further escalate to further people until addressed. The processor is designed to verify order fulfillment. The processor is further designed to have programmable guidelines wherein user errors are detected if guidelines are unmet.

In one embodiment of the healthcare product and service ordering system, the system further includes at least one or more guidelines wherein the processor can at least one or more of autonomously, by permission, and on demand at least one or more of correct and initiate correcting reasons for failing to meet the at least one guideline.

In one embodiment of the healthcare product and service ordering system, the system further includes at least one database adapted to collect at least one or more of data input, data process, data output, and corresponding movement of products and services.

In one embodiment of the healthcare product and service ordering system, the system further includes at least one machine learning program operationally coupled to the at least one database adapted to at least one or more of improve order timing, order processing, and guidelines and thresholds in at least one or more of real operations and simulations.

In one embodiment of the healthcare product and service ordering system, the system further includes at least one blockchain system adapted to add at least one hash to data units wherein data units can be traced to the origin of the data and the data verified.

In one embodiment of the healthcare product and service ordering system, the system further includes at least one blockchain system adapted to add at least one hash to received, modified, and created documents.

In one embodiment of the healthcare product and service ordering system, the system further includes adding at least one security code to authorize at least one or more of receiving, retrieving, modifying, and sending documents.

The inventive concept, including a corresponding method for using the disclosed system, now will be described more fully hereinafter with reference to the accompanying drawings, which are intended to be read in conjunction with both this summary, the detailed description, and any preferred and/or particular embodiments specifically discussed or otherwise disclosed. This inventive concept may, however, be embodied in many different forms and should not be construed as limited to the embodiments set forth herein; rather, these embodiments are provided by way of illustration only and so that this disclosure will be thorough, complete, and will fully convey the full scope of the inventive concept to those skilled in the art.

### BRIEF DESCRIPTION OF THE DRAWINGS

FIG. 1 illustrates a representative healthcare service order fulfillment system;

FIG. 2 illustrates a representative healthcare service order fulfillment method with system elements from FIG. 1;

FIG. 3 illustrates a representative computer processor;

FIG. 4 illustrates machine learning components of artificial intelligence;

FIG. 5 illustrates a representative blockchain transaction; and,

FIG. 6A-6F illustrate a representative healthcare service order fulfillment method.

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

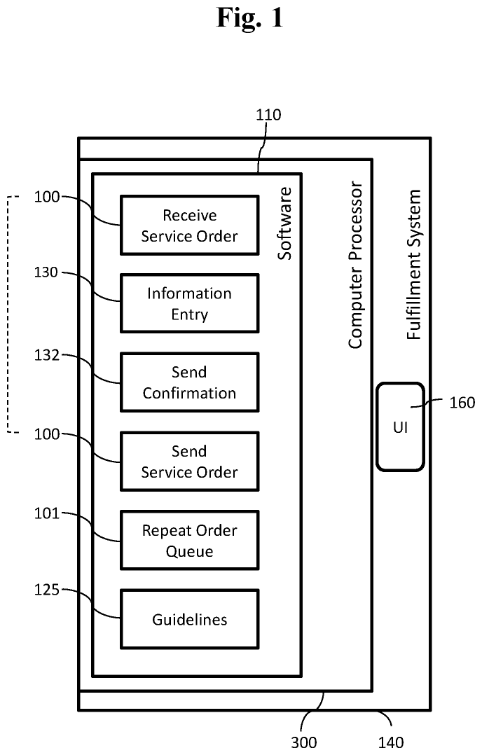

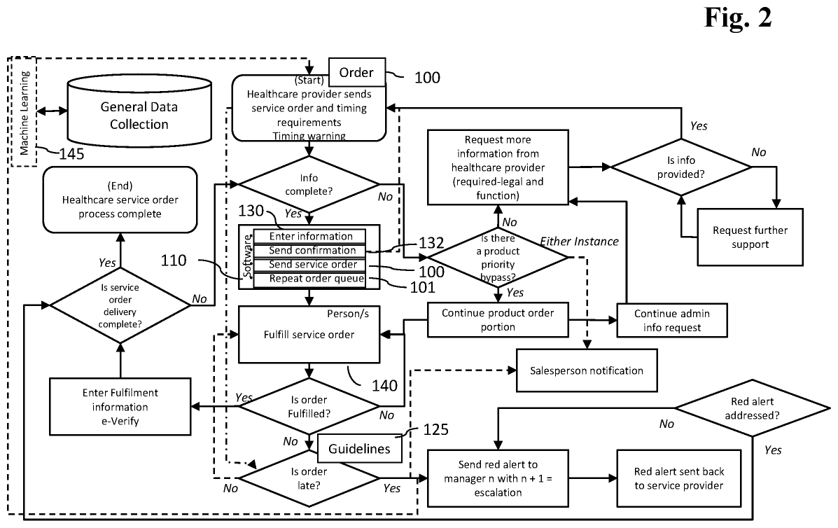

The inventive concept disclosed includes a healthcare product and service ordering system that has, as in one representative embodiment illustrated in FIGS. 1-3, at least one computer processor 340 of at least one computer system 300 adapted to receive an order 100 via at least one user interface 160 operable with at least one memory storage medium 330 wherein the service order 100 includes at least one or more of a product and a service. The at least one computer processor 340 is designed to assess the completeness of the order 100. The order 100 contains information 130 about at least one or more of the product and service ordered and associated administrative communications. The computer processor 340 is programmed to distinguish between the product and service ordered and associated administrative communications. A software program 110 is designed with components designed to at least one or more of record the information 130, send confirmation 132, send the order 100 for fulfillment 140, and establish a repeat order queue 101 wherein orders 100 may be repeated at least one or more of on a time schedule and on demand, wherein the software program 110 may be disposed on at least one or more of a single computer processor 340 and distributed system of computer processors 340. Machine learning 145 may further be used as will be further detailed.

FIGS. 1-3 further illustrate that the at least one computer processor 340 is designed to assess the priority of the at least one or more of a product and service ordered and, upon reaching a selectable priority bypass trigger point, is designed to select the order 100 for fulfillment 140. The at least one computer processor 340 is further designed to send incomplete information 130 to the order 100 source for completion. The at least one computer processor 340 is designed to receive for fulfillment 140 is at least one or more of the completed order information 130 from the software program 110 and the order 100 for the at least one or more product and service needed to fulfill the order 100. The computer processor 340 is designed to monitor order fulfillment 140 until fulfillment 140 is completed.

FIGS. 1-3 further illustrate that the computer processor 340 is designed to assess whether the order 100 is fulfilled on time. The computer processor 340, if fulfillment 140 is at least one or more of late, reaches a selected threshold prior to lateness, is determined will be late, and at least one or more of quantitatively, qualitatively, and by delivery location incomplete, is designed to alert at least one or more of a salesperson and a manager, designed to further escalate to further people until addressed. The computer processor 340 is designed to verify order 100 fulfillment 140. The computer processor 340 is further designed to have programmable guidelines 125 wherein user errors are detected if guidelines 125 are unmet.

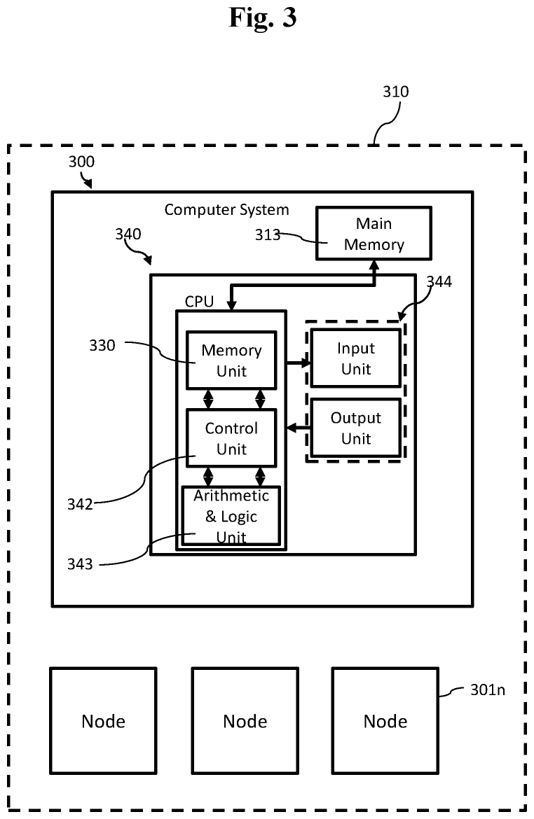

With reference to FIG. 3, the processor or central processing unit (CPU) 340, also called a central processor or main processor, is the electronic circuitry within the at least one computer system 300 that executes instructions that make up a computer program such as the disclosed software system 110. The CPU 340 performs basic arithmetic, logic, controlling, and input/output (I/O) operations specified by the instructions in the program. An arithmetic & logic unit (ALU) 343 is a combination digital electronic circuit that performs arithmetic and bitwise operations in integer binary numbers. Traditionally, the term CPU 340 refers to a processor, more specifically to its processing unit and control unit (CU) 342, distinguishing these core elements of a computer from external components such as main memory 313 and input output (I/O) circuitry 344. A CPU 340 may also contain memory 330. Memory 330 refers to a component that is used to store information 130 for immediate use in one or more computer systems 300 of a network 310, the network 310 which may have many nodes 301n.

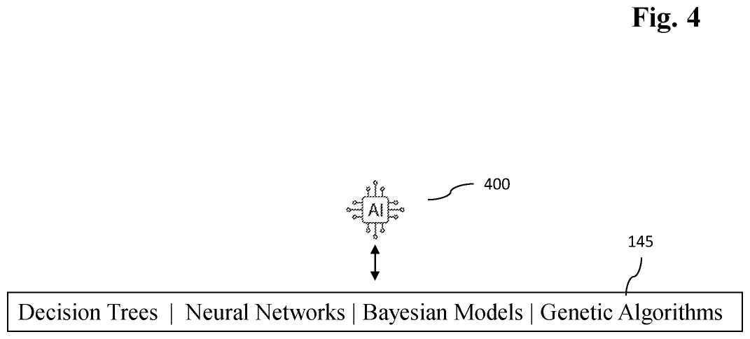

The healthcare product and service ordering system is designed to help users assess, decide, and act on orders and fulfillments proficiently faster than possible for traditional, often manual or mostly manual, ordering systems wherein the associated computer system 300 can at least reduce the opportunity for error and the ability for people to make decisions that work against the interest in the health of the patients. These reductions may be enhanced by, as illustrated in FIG. 4, artificial intelligence 400 at least one or more of using data in real time and simulating assessment, decision, and action cycles on user motions within the encoded framework of encoded software programs 110 therein, the programs which may further be enhanced by artificial intelligence 400.

Where artificial intelligence 400 is used, machine learning 145 may be employed to improve upon the at least one computer system's ability to assess, decide, and act on data collected from the inventive concept where improvement may be sought both on use of the data and the actions taken to fulfill orders.

Embodiments of the healthcare product and service ordering system may use decision tree strategies to determine which actions the computer system will take, for example, when to set thresholds for order 100 fulfillment timing. The healthcare product and service ordering system may employ for machine learning 145 one or more of decision trees, neural networks, Bayesian models, and genetic algorithms. The decision trees, neural networks, Bayesian models, and genetic algorithms may, in some embodiments, be derived from preset or partially preset libraries.

FIG. 4 further illustrates that the machine learning program 145 may support the healthcare product and service ordering system and an associated method that will follow and is designed to be used on a substantially continual cycle, at least one or more of assess data from the data about and generated by one or more orders, recommend actions as directed by the orders, with the further aim to improve interpretation of given actions, and choose how best to respond to commands. The machine learning program 145 adapts its performance to improve the accuracy of the at least one processor's 340 response for both the given order 100 and the aggregate of orders.

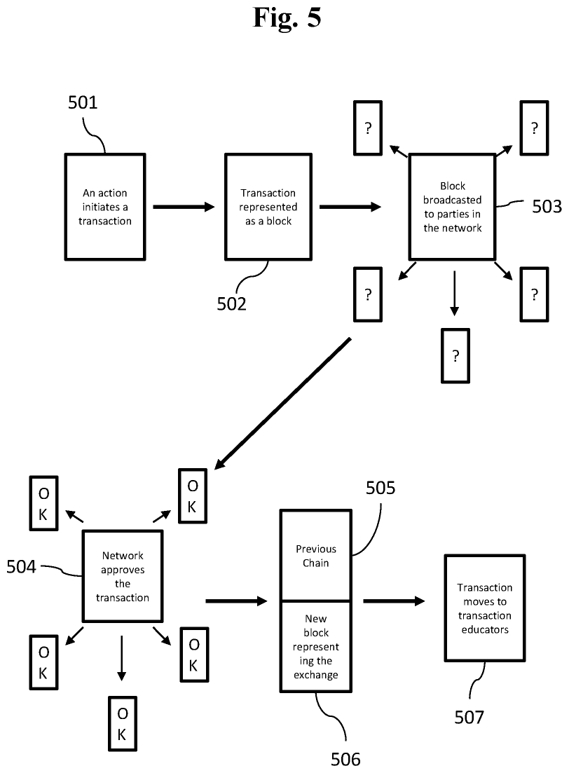

Now referring to FIG. 5, a blockchain update according to some implementations is shown. In step 501 of the process, an action initiates a transaction wherein the transaction may be the creation, receipt, modification, movement, or other event associated with at least one or more of data and data documentation along with transactions that may be a trade of value and information such as, but not limited to, money, instructions, and the actions involving receiving and fulfilling a service order 100. In step 502, the transaction is initiated in step 501 and is represented as a block. In some embodiments, the transaction may be compared with transaction records in the longest chain in the distributed system to verify the action. In some embodiments, a plurality of nodes in the network may compete to form the block containing the transaction record.

In some embodiments, nodes may be required to satisfy proof-of-work by solving a difficult mathematical problem to form the block. In some embodiments, other methods of proof such as proof-of-stake, proof-of-time, or proof-of-space, may be used in the system. In some embodiments, a block may represent one or more transactions that are broadcasted to the nodes 301n. In step 503, the block is broadcasted to parties in the network. In step 504, nodes in the network approve the exchange by examining the block that contains the aggregated transactions. In some embodiments, the nodes may check the solution provided as proof-of-work to approve the block. In some embodiments, the nodes may check the transaction against the transaction record in the longest blockchain in the system to verify that the transaction is valid reflecting a given action. In some embodiments, a block may be approved with consensus of the nodes 301n in the network 310. A transaction may be event driven. A transaction can, but is not required, to involve an exchange of monetary value.

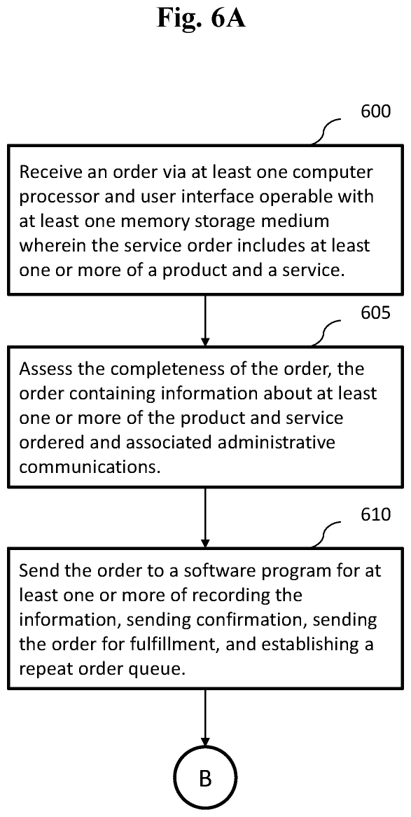

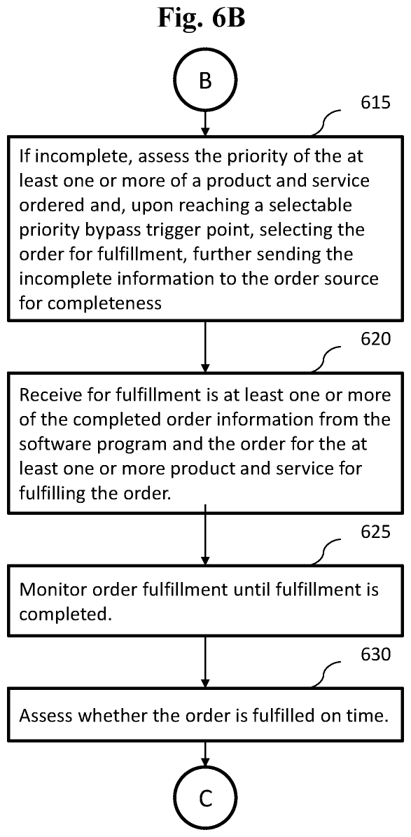

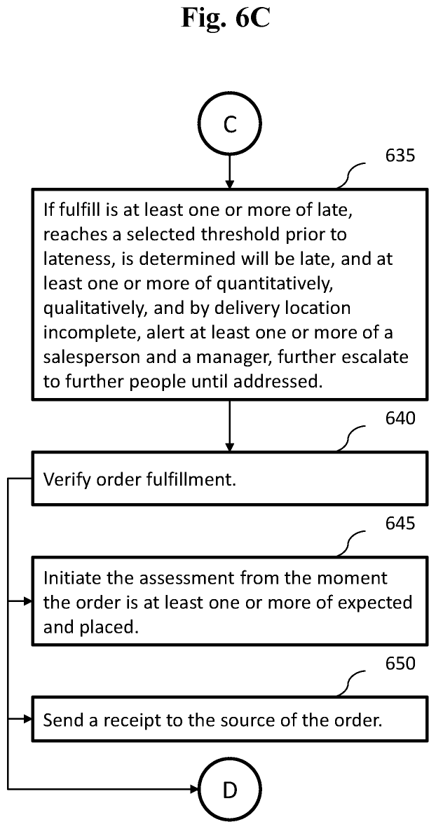

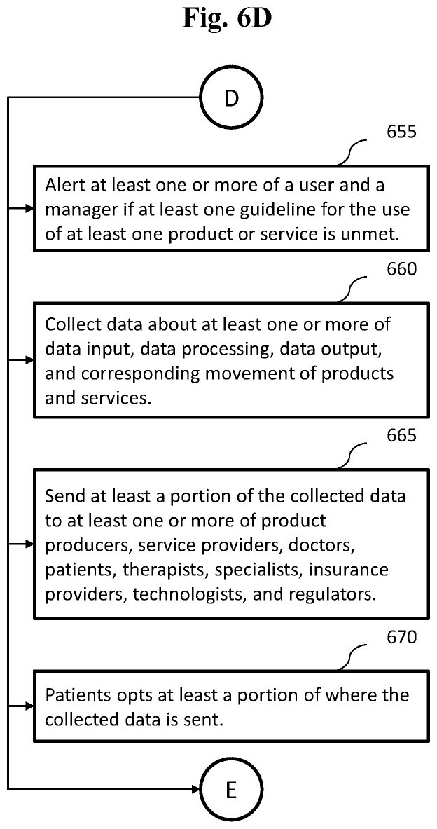

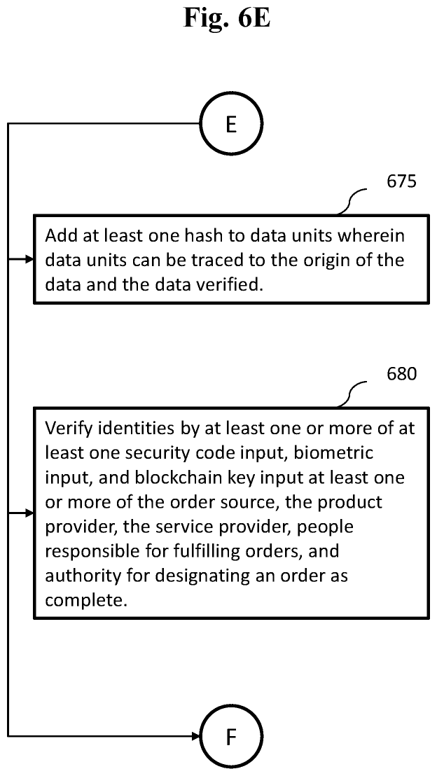

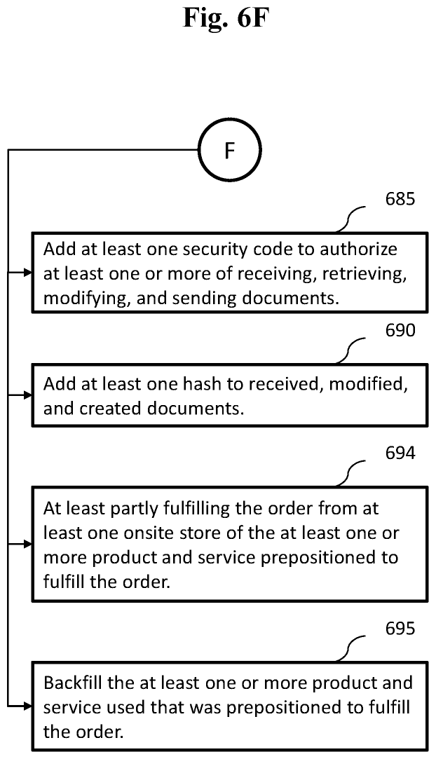

As illustrated in FIG. 6A-6F is a representative healthcare product and service ordering method, the method including 600 receiving an order 100 via at least one computer processor 340 and user interface operable with at least one memory storage medium 330 wherein the service order 100 includes at least one or more of a product and a service. The method further includes the step of 605, assessing the completeness of the order, the order 100 containing information 130 about at least one or more of the product and service ordered and associated administrative communications. The method further includes the step of 610, if complete, sending the order 100 to a software program 110 for at least one or more of recording the information 130, sending confirmation 132, sending the order 100 for fulfillment 140, and establishing a repeat order queue 101. The method further includes the step of 615, if incomplete, assessing the priority of the at least one or more of a product and service ordered and, upon reaching a selectable priority bypass trigger point, selecting the order 100 for fulfillment 140, further sending the incomplete information 130 to the order source for completion.

As illustrated in FIG. 6A-6F, the method further includes the step of 620, receiving for fulfillment 140 at least one or more of the completed order information 130 from the software program 110 and the order 100 for the at least one or more product and service needed to fulfill the order. The method further includes the step of 625, monitoring order fulfillment 140 until fulfillment is completed. The method further includes the step of 630, assessing whether the order 100 is fulfilled on time. The method further includes the step of 635, if fulfillment 140 is at least one or more of late, reaches a selected threshold prior to lateness, is determined will be late, and at least one or more of quantitatively, qualitatively, and by delivery location incomplete, alerting at least one or more of a salesperson and a manager, further escalating to further people until addressed. The method further includes the step of 640, verifying order fulfillment 140.

As illustrated in FIG. 6A-6F, the method may further include the step of 645, initiating the assessment of order fulfillment 140 from the moment the order 100 is at least one or more of expected and placed. The method may further include the step of 650, sending a receipt to the source of the order. The method may further include the step of 655, alerting at least one or more of a user and a manager if at least one guideline for the use of at least one product or service is unmet. The method may further include the step of 660, collecting data about at least one or more of data input, data processing, data output, and corresponding movement of products and services.

As illustrated in FIG. 6A-6F, the method may further include the step of 665, sending at least a portion of the collected data to at least one or more of, but not limited to, product producers, service providers, doctors, patients, therapists, specialists, insurance providers, technologists, and regulators. The method may further include the step of 670, the patients opting in to at least a portion of where the collected data is sent. The method may further include the step of 675, adding at least one hash to data units wherein data units can be traced to the origin of the data and the data verified.

As illustrated in FIG. 6A-6F, the method may further include the step of 680, verifying identities by at least one or more of at least one security code input, biometric input, and blockchain key input at least one or more of the order source, the product provider, the service provider, people responsible for fulfilling orders, and authority for designating an order 100 as complete.

As illustrated in FIG. 6A-6F, the method may further include the step of 685, adding at least one security code to authorize at least one or more of receiving, retrieving, modifying, and sending documents. The method may further include the step of 690, adding at least one hash to at least one or more of received, modified, and created documents. A transaction may include, but is not limited to, an exchange of documents, a review of documents, an alteration of documents, and a duplication of such documents, and the blockchain may further permit or prevent such transactions and provide the opportunity to audit such transactions back to their origin.

As illustrated in FIG. 6A-6F, the method may further include the step of 694, at least partly fulfilling the order 100 from at least one onsite store of the at least one or more product and service prepositioned to fulfill the order. The method may further include the step of 695, backfilling the at least one or more product and service used that was prepositioned to fulfill the order. Such is designed to allow orders to be fulfilled sooner by forward-deploying products and services.

Various related embodiments of the inventive concept are also described in Appendix A, which is incorporated herein by reference in its entirety. The following patents are incorporated by reference in their entireties: U.S. Pat. Nos. 6,356,874, 7,539,656, 7,860,750B2, 9,305,281B2, 9,536,049, and 9,824,188, U.S. Ser. Nos. 10/242,336B1, 10/607,275B2, 10/825,032B2, 11/029,918, and 11/087,386B2, US20020032591, US20050060217, US20120143619, US20140039962, US20150371237, US20170098185A1, US20190057340, US20210056516, CN106506881A, CN109726372A, CN109889392A, CN110225210A.
Order 100
Repeat order queue 101
Software program 110
Guidelines 125
Information 130
Fulfillment 140
User interface 160
Memory storage medium 330
Computer processor 340
Arithmetic & logic unit (ALU) 343
Control unit (CU) 342
Main memory 313
Input output (I/O) circuitry 344
Blockchain transaction process 501-504
Nodes 301n
Network 310
Artificial intelligence 400
Machine learning 145
Method 600-695

While the inventive concept has been described above in terms of specific embodiments, it is to be understood that the inventive concept is not limited to these disclosed embodiments. Upon reading the teachings of this disclosure, many modifications and other embodiments of the inventive concept will come to mind of those skilled in the art to which this inventive concept pertains, and which are intended to be and are covered by both this disclosure and the appended claims. It is indeed intended that the scope of the inventive concept should be determined by proper interpretation and construction of the appended claims and their legal equivalents, as understood by those of skill in the art relying upon the disclosure in this specification and the attached drawings.

1. A healthcare product and service ordering system comprising:
at least one computer processor adapted to receive an order via at least one user interface operable with at least one memory storage medium wherein the service order includes at least one or more of a product and a service;
the at least one processor adapted to assess the completeness of the order, the order containing information about at least one or more of the product and service ordered and associated administrative communications, the processor programmed to distinguish between the product and service ordered and associated administrative communications;
a software program adapted to at least one or more of record the information, send confirmation, send the order for fulfillment, and establish a repeat order queue, wherein the software program may be disposed on at least one or more of a single processor and distributed system;
the at least one processor adapted to assess the priority of the at least one or more of a product and service ordered and, upon reaching a selectable priority bypass trigger point, adapted to select the order for fulfillment, further adapted to send incomplete information to the order source for completion;
the at least one processor adapted to receive for fulfillment at least one or more of the completed order information from the software program and the order for the at least one or more product and service needed to fulfill the order;
the processor adapted to monitor order fulfillment until fulfillment is completed;
the processor adapted to assess whether the order is fulfilled on time;
the processor, if fulfillment is at least one or more of late, reaches a selected threshold prior to lateness, is determined will be late, and at least one or more of quantitatively, qualitatively, and by delivery location incomplete, adapted to alert at least one or more of a salesperson and a manager, adapted to further escalate to further people until addressed;
the processor further adapted to verify order fulfillment; and
the processor further adapted to have programmable guidelines wherein user errors are detected if guidelines are unmet.

2. The healthcare product and service ordering system of claim 1, the system further including at least one or more guidelines wherein the processor can at least one or more of autonomously, by permission, and on demand at least one or more of correct and initiate correcting reasons for failing to meet the at least one guideline.

3. The healthcare product and service ordering system of claim 1, the system further including at least one database adapted to collect at least one or more of data input, data process, data output, and corresponding movement of products and services.

4. The healthcare product and service ordering system of claim 3, the system further including at least one machine learning program operationally coupled to the at least one database adapted to at least one or more of improve order timing, order processing, and guidelines and thresholds in at least one or more of real operations and simulations.

5. The healthcare product and service ordering system of claim 3, the system further including at least one blockchain system adapted to add at least one hash to data units wherein data units can be traced to the origin of the data and the data verified.

6. The healthcare product and service ordering system of claim 1, the system further including at least one blockchain system adapted to add at least one hash to received, modified, and created documents.

7. The healthcare product and service ordering system of claim 1, the system further including adding at least one security code to authorize at least one or more of receiving, retrieving, modifying, and sending documents.

8. A healthcare product and service ordering method comprising:
receiving an order via at least one computer processor and user interface operable with at least one memory storage medium wherein the service order includes at least one or more of a product and a service;
assessing the completeness of the order, the order containing information about at least one or more of the product and service ordered and associated administrative communications;
if complete, sending the order to a software program for at least one or more of recording the information, sending confirmation, sending the order for fulfillment, and establishing a repeat order queue;
if incomplete, assessing the priority of the at least one or more of a product and service ordered and, upon reaching a selectable priority bypass trigger point, selecting the order for fulfillment, further sending the incomplete information to the order source for completion;
receiving for fulfillment at least one or more of the completed order information from the software program and the order for the at least one or more product and service needed to fulfill the order;
monitoring order fulfillment until fulfillment is completed;
assessing whether the order is fulfilled on time;
if fulfillment is at least one or more of late, reaches a selected threshold prior to lateness, is determined will be late, and at least one or more of quantitatively, qualitatively, and by delivery location incomplete, alerting at least one or more of a salesperson and a manager, further escalating to further people until addressed; and
verifying order fulfillment.

9. The healthcare product and service ordering method of claim 8, the method further including initiating the assessment from the moment the order is at least one or more of expected and placed.

10. The healthcare product and service ordering method of claim 8, the method further including sending a receipt to the source of the order.

11. The healthcare product and service ordering method of claim 8, the method further including alerting at least one or more of a user and a manager if at least one guideline for the use of at least one product or service is unmet.

12. The healthcare product and service ordering method of claim 8, the method further including collecting data about at least one or more of data input, data processing, data output, and corresponding movement of products and services.

13. The healthcare product and service ordering method of claim 12, the method further including sending at least a portion of the collected data to at least one or more of product producers, service providers, doctors, patients, therapists, specialists, insurance providers, technologists, and regulators.

14. The healthcare product and service ordering method of claim 13, the method further including the patients opting in to for at least a portion of where the collected data is sent.

15. The healthcare product and service ordering method of claim 12, the method further including adding at least one hash to data units wherein data units can be traced to the origin of the data and the data verified.

16. The healthcare product and service ordering method of claim 8, the method further including verifying identities by at least one or more of at least one security code input, biometric input, and blockchain key input at least one or more of the order source, the product provider, the service provider, people responsible for fulfilling orders, and authority for designating an order as complete.

17. The healthcare product and service ordering method of claim 8, the method further including adding at least one security code to authorize at least one or more of receiving, retrieving, modifying, and sending documents.

18. The healthcare product and service ordering method of claim 8, the method further including adding at least one hash to at least one or more of received, modified, and created documents.

19. A healthcare product and service ordering method comprising:
receiving an order via at least one computer processor and user interface operable with at least one memory storage medium wherein the service order includes at least one or more of a product and a service;
assessing the completeness of the order, the order containing information about at least one or more of the product and service ordered and associated administrative communications;
if complete, sending the order to a software program for at least one or more of recording the information, sending confirmation, sending the order for fulfillment, and establishing a repeat order queue;
if incomplete, assessing the priority of the at least one or more of a product and service ordered and, upon reaching a selectable priority bypass trigger point, selecting the order for fulfillment, further sending the incomplete information to the order source for completion;
receiving for fulfillment at least one or more of the completed order information from the software program and the order for the at least one or more product and service needed to fulfill the order;
at least partly fulfilling the order from at least one onsite store of the at least one or more product and service prepositioned to fulfill the order;
monitoring order fulfillment until fulfillment is completed;
monitoring prepositioned products and services for viability and availability;
assessing whether the order is fulfilled on time;
if fulfillment is at least one or more of late, reaches a selected threshold prior to lateness, is determined will be late, and at least one or more of quantitatively, qualitatively, and by delivery location incomplete, alerting at least one or more of a salesperson and a manager, further escalating to further people until addressed;
backfilling the at least one or more product and service used that was prepositioned to fulfill the order; and
verifying order fulfillment.

20. The healthcare product and service ordering method of claim 19, the method further including adding at least one hash to data units wherein data units can be traced to the origin of the data and the data verified.
